(19) United States
(12) Patent Application Publication
(10) Pub. No.: US 2026/0071171 A1
(43) Pub. Date: Mar. 12, 2026

(54) Mycelium Structure And Method For Producing Mycelium Structure

(71) Applicant: SEIKO EPSON CORPORATION, Tokyo (JP)

(72) Inventor: Tomohiro HAYASHI, Matsumoto (JP)

(21) Appl. No.: 19/320,400

(22) Filed: Sep. 5, 2025

(30) Foreign Application Priority Data
Sep. 6, 2024 (JP) 2024-153789

(51) Int. Cl. C12N 1/14

(52) U.S. Cl. CPC C12N 1/14; C12N 2513/00; C12N 2533/70

(57) ABSTRACT

A mycelium structure according to the present disclosure includes: a mycelium formed of hyphae connected in a three-dimensional shape; and starch covering the hyphae. A density of the mycelium structure is preferably 0.8 g/cm³ or more and 5.0 g/cm³ or less. An average diameter of the hyphae covered with the starch is preferably 2.0 μm or more and 20.0 μm or less. In a stress-strain curve indicating a relationship between a stress (MPa) applied by a tensile test and a strain (%) at that time, a maximum stress in an elastic region is preferably 2.0 MPa or more and 40.0 MPa or less, and a maximum strain is preferably 0.5% or more and 4.0% or less.

The present application is based on, and claims priority from JP Application Serial Number 2024-153789, filed Sep. 6, 2024, the disclosure of which is hereby incorporated by reference herein in its entirety.

### BACKGROUND

### 1. Technical Field

The present disclosure relates to a mycelium structure and a method for producing a mycelium structure.

### 2. Related Art

In recent years, there has been a growing demand in a market for products that use natural materials and have a low environmental load. For example, JP-A-2003-201695 discloses a composite material in which a solid content of 65 wt % to 100 wt % is formed of cellulose microfibrils and high strength is ensured.

JP-A-2003-201695 is an example of the related art.

Although compatibility between strength and flexibility is required in order to use a material for various uses, there is a problem that the composite material in JP-A-2003-201695 has high strength and poor flexibility. Therefore, it is required to provide a mycelium structure as a material having excellent mechanical strength and flexibility.

### SUMMARY

A mycelium structure according to an application example of the present disclosure includes: a mycelium formed of hyphae connected in a three-dimensional shape; and starch covering the hyphae.

A method for producing a mycelium structure according to an application example of the present disclosure includes: a permeation step of causing a starch solution to permeate into a mycelium obtained by culture; and a drying step of drying the mycelium permeated with the starch solution.

### BRIEF DESCRIPTION OF THE DRAWINGS

FIG. 1 is a process diagram showing a configuration of a method for producing a mycelium structure according to an embodiment.

FIG. 2 is Table 1 showing configurations of mycelium structures and evaluation results of the mycelium structure in Examples and Comparative Example.

### DESCRIPTION OF EMBODIMENTS

Hereinafter, a mycelium structure and a method for producing a mycelium structure according to the present disclosure will be described in detail based on preferred embodiments illustrated in the accompanying drawings.

### [1] Mycelium Structure

First, the mycelium structure according to an embodiment will be described.

The mycelium structure includes: a mycelium formed of hyphae connected in a three-dimensional shape; and starch covering the hyphae. According to such a configuration, a mycelium structure excellent in mechanical strength and flexibility can be obtained.

### [1-1] Mycelium

The mycelium contains at least mushroom hyphae. Accordingly, the flexibility of the mycelium structure can be made excellent.

The mushroom hyphae have a fibrous structure constituting a mycelium of mushroom. A type of the mushroom is not particularly limited, and examples thereof include Agaricus arvensis, Agrocybe brasiliensis, Amylomyces rouxii, species of Amylomyces, Armillaria mellea, Aspergillus nidulans, Aspergillus niger, Aspergillus oryzae, Ceriporia lacerata, Coprinus comatus, Fibroporia vaillantii, Fistulina hepatica, Flammulina velutipes, Fomitopsis officinalis, Ganoderma sessile, Ganoderma tsugae, Ganoderma lucidum, Hericium erinaceus, Hypholoma capnoides, Hypholoma sublaterium, Inonotus obliquus, Lactarius chrysorrheus, Macrolepiota procera, Morchella angusticeps, Myceliophthora thermophila, Neurospora crassa, Penicillium camembertii, Penicillium chrysogenum, Penicillium rubens, Phycomyces blakesleeanus, Pleurotus djamor, Pleurotus ostreatus, Polyporus squamosus, Psathyrella aquatica, Rhizopus microspores, Rhizopus oryzae, Schizophyllum commune, Streptomyces venezuelae, Stropharia rugosoannulata, Thielavia terrestris, Ustilago maydis, Shiitake mushroom (Lentinula genus), Meripilus giganteus (Meripilus genus), Grifola frondosa (Grifola genus), Leucopaxillus giganteus (Leucopaxillus genus), polyporaceae (Fomitopsis genus), and Tricholoma matsutake (Tricholoma genus).

The mycelium is an aggregation of multiple mushroom hyphae. In the following description, the mushroom hyphae are also simply referred to as “hyphae”.

An average diameter of hyphae constituting the mycelium is preferably 0.2 μm or more and 4.0 μm or less, more preferably 0.3 μm or more and 3.5 μm or less, and still more preferably 0.4 μm or more and 3.0 μm or less. In the present disclosure, “hyphae constituting mycelium” refers to hyphae before being covered with starch described later. When the average diameter of hyphae constituting the mycelium is within the above range, a mycelium structure having particularly improved mechanical strength and flexibility can be produced.

The average diameter of hyphae constituting the mycelium is measured as follows.

First, a mycelium is magnified and observed so that 100 or more hyphae are contained in one image, and an image is acquired. Next, 10 or more hyphal images are randomly selected, and a width of a hyphal image is measured. An average value of measured values is the average diameter of hyphae constituting the mycelium. An average diameter of hyphae covered with starch described later is also measured in the same manner.

An average length of the hyphae constituting the mycelium is not particularly limited, and is preferably 0.01 mm or more and 3.0 mm or less, more preferably 0.10 mm or more and 2.0 mm or less, and still more preferably 0.50 mm or more and 1.0 mm or less. When the average length of the hyphae constituting the mycelium is within the above range, for example, when the mycelium structure is formed into a sheet shape, the hyphae are oriented along a surface of the mycelium structure and the hyphae are moderately entangled with each other. Accordingly, mechanical strength and flexibility of the mycelium structure can be particularly enhanced.

The average length of hyphae constituting the mycelium is measured as follows.

First, a mycelium is magnified and observed so that 100 or more hyphae are contained in one image, and an image is acquired. Next, 10 or more hyphal images are randomly selected, and a maximum possible length in the hyphal image is measured. An average value of measured values is the average length of the hyphae constituting the mycelium. An average length of hyphae covered with starch described later is also measured in the same manner.

A fiber density of the hyphae constituting the mycelium is preferably 0.1 g/cm³ or more and 0.8 g/cm³ or less, more preferably 0.2 g/cm³ or more and 0.7 g/cm³ or less, and still more preferably 0.3 g/cm³ or more and 0.6 g/cm³ or less. When the fiber density of the hyphae constituting the mycelium is within the above range, the mechanical strength and flexibility of the mycelium structure can be particularly enhanced.

The fiber density of the hyphae constituting the mycelium is measured as follows.

First, a weight of mycelium is measured with an electronic balance. Next, a volume of mycelium is measured. The volume is an apparent volume including pores. By dividing a mass by the volume, the fiber density of the hyphae constituting the mycelium can be obtained. A fiber density of hyphae covered with starch described later is also measured in the same manner.

The hyphae preferably contain chitin. The chitin is contained as a component of a cell wall constituting the hyphae. The chitin is a high-molecular polysaccharide having N-acetylglucosamine in which an acetamide group is added to glucose as a structural unit. Since the chitin has a hydroxyl group, for example, when a crosslinking agent described later is used for imparting strength, the hyphae are easily crosslinked by the crosslinking agent. Accordingly, the mechanical strength of the mycelium structure can be further improved.

### [1-2] Starch

In the mycelium structure, at least a part of hyphae is covered with starch. Accordingly, the mechanical strength of the mycelium structure becomes excellent. In the following description, the starch is also simply referred to as “starch”.

The starch is a molecule obtained by polymerizing a plurality of α-glucose molecules through glycosidic bonds. The starch may be a linear molecule or may contain a branch. As the starch, it is possible to use, for example, starch derived from various plants. More specifically, it is possible to use, for example, a material derived from cereals such as corn, wheat, and rice, beans such as broad beans, mung beans, and adzuki beans, tubers such as potato, sweet potato, and tapioca, wild plants such as bracken and kudzu, and palm trees such as sago palm.

The starch may be modified starch. Examples of the modified starch include acetylated adipic acid crosslinked starch, acetylated starch, oxidized starch, sodium octenyl succinate, hydroxypropyl starch, hydroxypropylated phosphate crosslinked starch, phosphorylated starch, phosphate-esterified phosphate crosslinked starch, urea phosphate-esterified starch, sodium starch glycolate, and high amylose corn starch. The starch may be modified starch. Examples of the modified starch include those obtained by processing or modifying starch, and specific examples thereof include dextrin.

The average diameter of the hyphae covered with starch is preferably 2.0 μm or more and 20.0 μm or less, more preferably 3.0 μm or more and 18.0 μm or less, and still more preferably 4.0 μm or more and 15.0 μm or less. When the average diameter of hyphae covered with starch is within the above range, the mechanical strength and flexibility of the mycelium structure can be further improved.

A thickness of the starch covering the hyphae is preferably 1.8 μm or more and 16.0 μm or less, more preferably 2.7 μm or more and 14.5 μm or less, and still more preferably 3.6 μm or more and 11.0 μm or less. When the thickness of the starch covering the hyphae is within the above range, the mechanical strength and flexibility of the mycelium structure can be further improved. The thickness of the starch covering hyphae is a value obtained by subtracting the average diameter of hyphae constituting mycelium from the average diameter of hyphae covered with starch and dividing the result by 2.

The average diameter of hyphae constituting mycelium is D1 (μm), and the average diameter of hyphae covered with starch is D2 (μm). At this time, D2/D1 is a ratio of the average diameter after and before being covered with starch. D2/D1 is preferably 1.1 or more and 20.0 or less, more preferably 1.5 or more and 15.0 or less, and still more preferably 2.0 or more and 10.0 or less. By setting the ratio of D2/D1 within the above range, the mechanical strength and flexibility of the mycelium structure can be particularly enhanced.

A density of the mycelium structure is preferably 0.8 g/cm³ or more and 5.0 g/cm³ or less, more preferably 1.2 g/cm³ or more and 4.5 g/cm³ or less, and still more preferably 1.5 g/cm³ or more and 4.0 g/cm³ or less. When the density of the mycelium structure is within the above range, the mechanical strength and flexibility of the mycelium structure can be particularly improved.

The porosity of the mycelium structure is not particularly limited, and is preferably 1.0% by volume or more and 20.0% by volume or less, more preferably 2.0% by volume or more and 15.0% by volume or less, and still more preferably 3.0% by volume or more and 10.0% by volume or less.

The fiber density of the hyphae constituting the mycelium is M1 (g/cm³), and the density of the mycelium structure is M2 (g/cm³). At this time, M2/M1 is a mass ratio before and after being covered with starch. M2/M1 is preferably 2.0 or more and 20.0 or less, more preferably 2.5 or more and 15.0 or less, and still more preferably 3.0 or more and 10.0 or less. By setting the ratio of M2/M1 within the above range, the mechanical strength and flexibility of the mycelium structure can be particularly enhanced.

### [1-3] Other Components Contained in Mycelium

The mycelium may contain other components. Examples of the other components include a plasticizer, a stabilizer, an antioxidant, an ultraviolet absorber, a lubricant, a flame retardant, an antistatic agent, a colorant, and a filler.

Examples of the plasticizer include oils, sugar alcohols, glycerin, adipic acid ester-based plasticizers, phthalic acid ester-based plasticizers, trimellitate-based plasticizers, polyester-based plasticizers, (meth)acrylic acid ester polymers, ethylene copolymer elastomers, chlorinated polyethylene (CPE), (meth)acrylic resins (PMMA), polystyrene resins (PS), polyvinyl acetate resins (PVAc), acrylonitrile-butadiene rubber (NBR), and styrene-butadiene rubber (SBR). When the mycelium contains a plasticizer, the flexibility of the mycelium structure can be enhanced.

The oil refers to a hydrophobic liquid, and is generally an ester of an alcohol and a fatty acid. Examples of the oil include plant-derived, animal-derived, and mineral-derived oils. Examples of the vegetable oil include castor oil, rapeseed oil, soybean oil, palm oil, linseed oil, olive oil, avocado oil, sesame oil, perilla oil, cottonseed oil, safflower oil, corn oil, rice bran oil, camellia oil, coconut oil, and peanut oil. Specific examples thereof include epoxidized vegetable oils such as epoxidized soybean oil (ESBO) and epoxidized linseed oil (ELSO).

Examples of the sugar alcohol include maltitol, lactitol, tetritol, pentitol, hexitol, erythritol, sorbitol, xylitol, mannitol, and glycerin.

A content of the other components in the mycelium is not particularly limited, and is preferably 20.0% by mass or less, more preferably 15.0% by mass or less, and still more preferably 10.0% by mass or less.

### [1-4] Others

Substances contained in the mycelium may be crosslinked with each other by a crosslinking agent. The crosslinking agent reacts with the hydroxyl groups contained in hyphae and fiber when heat is applied. Accordingly, the mechanical strength of the mycelium structure can be improved.

Examples of the crosslinking agent include aliphatic dicarboxylic acids such as oxalic acid, malonic acid, succinic acid, glutaric acid, adipic acid, pimelic acid, suberic acid, azelaic acid, and sebacic acid; aromatic dicarboxylic acids such as phthalic acid, isophthalic acid, and terephthalic acid; dicarboxylic acids having a hydroxyl group such as tartaric acid and malic acid; tricarboxylic acids such as citric acid and aconitic acid; and amino acids having a plurality of carboxy groups such as aspartic acid and glutamic acid. One or a mixture of two or more of these may be used.

The mycelium may be treated by an ozone treatment, a deacetylation treatment, sebacic acid, tannin, or the like to change a chemical bond of the mycelium. These treatments can further improve the mechanical strength of the mycelium structure.

Treatments for improving the mechanical strength and flexibility of the mycelium structure may be performed alone or in combination as appropriate.

### [1-5] Use of Mycelium Structure

The mycelium structure can be formed into various shapes according to, for example, its use. More specifically, for example, it can be formed into a sheet shape, a board shape, or a web shape. Specific examples of the use include paper, nonwoven fabric, wallpaper, wrapping paper, colored paper, drawing paper, recording medium, decorative sheet, fiber board, filter, liquid absorbent, sound absorber, cushioning material, and mat.

Since the mycelium structure according to the embodiment is excellent in mechanical strength and flexibility, the mycelium structure is particularly useful as a naturally derived material such as a leather substitute material (substitute leather).

### [1-6] Features of Mycelium Structure

In the mycelium structure according to the embodiment, in a stress-strain curve indicating a relationship between the stress (MPa) applied by a tensile test and a strain (%) at that time, a maximum stress in an elastic region is preferably 2.0 MPa or more and 40.0 MPa or less, more preferably 5.0 MPa or more and 35.0 MPa or less, and still more preferably 8.0 MPa or more and 30.0 MPa or less. Accordingly, the mechanical strength of the mycelium structure can be further enhanced.

In the mycelium structure according to the embodiment, stress-strain curve indicating the relationship between the stress (MPa) applied by the tensile test and the strain (%) at that time, a maximum strain in the elastic region is preferably 0.5% or more and 4.0% or less, more preferably 1.0% or more and 3.5% or less, and still more preferably 1.5% or more and 3.0% or less. Accordingly, the flexibility of the mycelium structure can be particularly improved.

For the tensile test, for example, Autograph AGS-5kNX (manufactured by Shimadzu Corporation) can be used.

### [2] Method for Producing Mycelium Structure

Next, an example of the method for producing a mycelium structure described above will be described.

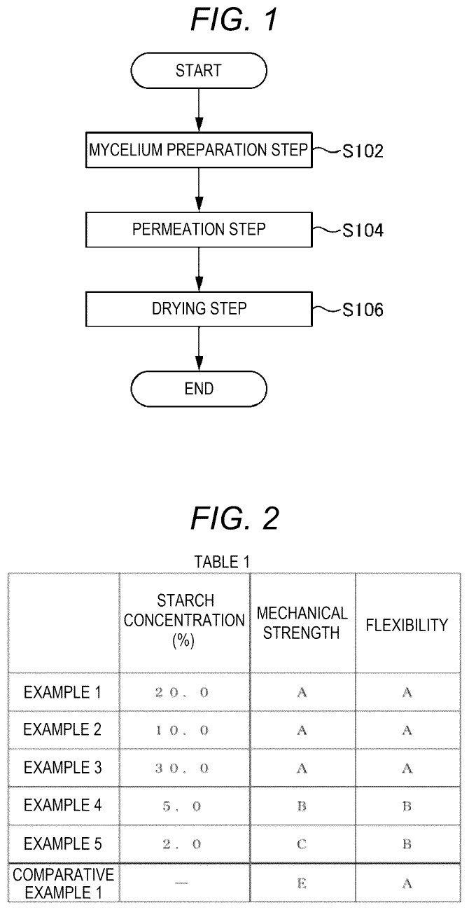

FIG. 1 is a process diagram showing a configuration of the method for producing a mycelium structure according to the embodiment.

The method for producing a mycelium structure shown in FIG. 1 includes a mycelium preparation step S102 for preparing a mycelium, a permeation step S104 for causing a starch solution to permeate into the mycelium, and a drying step S106 for drying the permeated starch solution. According to such a configuration, a mycelium structure excellent in mechanical strength and flexibility can be produced.

### [2-1] Mycelium Preparation Step

In the mycelium preparation step S102, a mycelium containing hyphae is first prepared. The mycelium is formed, for example, by collecting a large number of hyphae and forming or paper-making the hyphae into a sheet. The mycelium may have a flat plate shape or may be formed into a predetermined shape.

As the mycelium, mycelium obtained by culture are preferably used. An average length of hyphae of the mycelium obtained by culture is longer than that of a defibrated material of mushroom mycelium. Therefore, in the mycelium obtained by culture, the mycelia are entangled with each other, so that the flexibility of the mycelium structure can be improved.

When inoculum of the hyphae is inoculated into the culture medium and cultured, a mycelium in which the grown hyphae are spread over the entire culture medium can be obtained. The culture medium may be a solid culture medium or a liquid culture medium.

The culture medium may have a component that improves mechanical strength and flexibility. Examples of the component that improves the mechanical strength and the flexibility include a plasticizer and a crosslinking agent as described above. In addition to these components, the culture medium may contain a nutrient, a gelling agent, and the like necessary for growth of mushroom hyphae.

In a case of a solid culture medium, a culture medium formed into a sheet shape may be used. Accordingly, secondary processing can be simplified or omitted, and the sheet-shaped mycelium can be efficiently produced. In the liquid culture medium, for example, nutrients, plasticizers, crosslinking agents, and the like are dispersed in a dispersion medium such as water. When a liquid culture medium is used, the medium is relatively easy to manage and handle because the culture medium is liquid. In the liquid culture medium, since an operation such as stirring is possible, it is easy to make culture uniform and speed up.

Culture conditions such as a culture temperature, a culture time, and humidity are appropriately set in accordance with a type of the hyphae or the culture medium.

In the mycelium obtained by culture, hyphae are three-dimensionally connected to each other. Accordingly, it is possible to impart better flexibility to the mycelium.

The mycelium obtained by culture may be formed as necessary. Accordingly, a mycelium having a desired shape can be obtained.

As the mycelium, a mycelium subjected to a treatment for improving mechanical strength and flexibility may be prepared. Examples of the treatment for improving the mechanical strength and flexibility include addition of a plasticizer and a crosslinking agent, and ozone treatment.

### [2-2] Permeation Step

Next, in the permeation step S104, the prepared mycelium is permeated into a starch solution. As a permeation method, a method for immersing the mycelium in a starch solution is preferable. The starch solution may permeate into at least a part of the mycelium, and it is preferable that the starch solution permeates into the entire mycelium.

The starch solution is a solution containing starch and water. A starch concentration in the starch solution is preferably 3.0% by mass or more and 30.0% by mass or less, and more preferably 5.0% by mass or more and 28.0% by mass or less. When the starch concentration is within the above range, the starch solution can more efficiently permeate into the mycelium.

The starch concentration in the starch solution is more preferably 15.0% by mass or more and 25.0% by mass or less, and particularly preferably 18.0% by mass or more and 22.0% by mass or less. When the starch concentration is within the above range, a range of the maximum stress and the maximum strain described above can be more easily satisfied. Accordingly, the mechanical strength and the flexibility of the mycelium structure can be further enhanced.

Any additive may be added to the starch solution as necessary. Examples of the additive include a condensing agent, an antioxidant, a stabilizer, and a lubricant.

A time for immersing the mycelium in the starch solution is preferably 130 minutes or longer, more preferably 145 minutes or longer, and still more preferably 160 minutes or longer. Accordingly, the starch solution can be permeated into the mycelium, and the mechanical strength and flexibility of the mycelium structure can be particularly enhanced.

The time for immersing the mycelium in the starch solution is preferably 250 minutes or shorter, more preferably 220 minutes or shorter, and still more preferably 200 minutes or shorter, from the viewpoint of productivity.

When the starch solution is heated at the time of permeation, a time until the starch solution permeates into the entire mycelium can be shortened. By decreasing viscosity of the starch solution, the time required for permeation can be shortened. By stirring the starch solution or applying an external force to the mycelium, a permeation time can be shortened by promoting a contact between the starch solution and the mycelium.

As the permeation method, vacuum impregnation may be used. In this case, by adjusting a degree of vacuum during impregnation and a vacuum time, it is possible to adjust a time required for permeation.

### [2-3] Drying Step

In the drying step S106, the mycelium permeated with the starch solution is dried to evaporate moisture. When the moisture evaporates, the starch coats the hyphae so as to gather around the hyphae. Accordingly, the mechanical strength and the flexibility of the mycelium structure can be enhanced.

As a drying condition, heating is preferably performed at 80.0° C. or higher and 120.0° C. or lower, more preferably 85.0° C. or higher and 115.0° C. or lower, and still more preferably 80.0° C. or higher and 110.0° C. or lower. Accordingly, moisture is further evaporated from the mycelium, and the mechanical strength and flexibility of the mycelium structure can be particularly enhanced. However, the drying condition is not limited to the above range, and it may be, for example, dried at room temperature.

The drying may be performed under atmospheric pressure, or may be performed under pressurization conditions. In this case, the pressurization condition is preferably 40.0 kPa or more and 230.0 kPa or less, more preferably 45.0 kPa or more and 225.0 kPa or less, and still more preferably 50.0 kPa or more and 220.0 kPa or less. Accordingly, moisture is further evaporated from the mycelium, and the mechanical strength and flexibility of the mycelium structure can be improved.

After the drying step, the permeation step and the drying step may be performed again. Accordingly, a starch coating on the mycelium can be made thicker, and the mechanical strength of the mycelium structure can be particularly improved.

### [3] Effects of Embodiment

As described above, the mycelium structure according to the embodiment includes: a mycelium formed of hyphae connected in a three-dimensional shape; and starch covering the hyphae.

According to such a configuration, a mycelium structure excellent in mechanical strength and flexibility can be obtained.

In the mycelium structure according to the embodiment, the density of the mycelium structure is preferably 0.8 g/cm³ or more and 5.0 g/cm³ or less.

According to such a configuration, the mechanical strength and flexibility of the mycelium structure can be particularly improved.

In the mycelium structure according to the embodiment, an average diameter of hyphae covered with starch is preferably 2.0 μm or more and 20.0 μm or less.

According to such a configuration, when the hyphae are covered with starch, the mechanical strength and flexibility of the mycelium structure can be further improved.

In the mycelium structure according to the embodiment, in the stress-strain curve indicating the relationship between the stress (MPa) applied by the tensile test and the strain (%) at that time, the maximum stress in the elastic region is preferably 2.0 MPa or more and 40.0 MPa or less.

According to such a configuration, the mechanical strength of the mycelium structure can be further enhanced.

In the mycelium structure according to the embodiment, in the stress-strain curve indicating the relationship between the stress (MPa) applied by the tensile test and the strain (%) at that time, the maximum strain in the elastic region is preferably 0.5% or more and 4.0% or less.

According to such a configuration, the flexibility of the mycelium structure can be particularly improved.

The method for producing a mycelium structure according to the embodiment includes: the permeation step S104 of causing a starch solution to permeate into a mycelium obtained by culture; and the drying step S106 of drying the mycelium permeated with the starch solution.

According to such a configuration, a mycelium structure excellent in mechanical strength and flexibility can be produced.

In the method for producing a mycelium structure according to the embodiment, in the drying step S106, the mycelium permeated with the starch solution is preferably heated at 80.0° C. or higher and 120.0° C. or lower.

According to such a configuration, moisture is further evaporated from the mycelium, and a mycelium structure having particularly improved mechanical strength and flexibility can be produced.

In the method for producing a mycelium structure according to the embodiment, in the drying step S106, the mycelium permeated with the starch solution is preferably pressurized at 40.0 kPa or more and 230.0 kPa or less.

According to such a configuration, moisture is further evaporated from the mycelium, and the mechanical strength and flexibility of the mycelium structure can be improved.

In the method for producing a mycelium structure according to the embodiment, an average diameter of the hyphae constituting the mycelium is preferably 0.2 μm or more and 4.0 μm or less.

According to such a configuration, a mycelium structure having particularly improved mechanical strength and flexibility can be produced.

In the method for producing a mycelium structure according to the embodiment, the fiber density of the hyphae constituting the mycelium is preferably 0.1 g/cm³ or more and 0.8 g/cm³ or less.

According to such a configuration, the mechanical strength and flexibility of the mycelium structure can be particularly enhanced.

In the method for producing a mycelium structure according to the embodiment, a starch concentration in the starch solution is preferably 3.0% by mass or more and 30.0% by mass or less.

According to such a configuration, it is possible to more efficiently permeate the starch solution into an inside of the mycelium, and it is possible to produce a mycelium structure having particularly improved mechanical strength and flexibility.

In the method for producing a mycelium structure according to the embodiment, the starch concentration in the starch solution is preferably 15.0% by mass or more and 25.0% by mass or less.

According to such a configuration, it is possible to more easily satisfy the range of the maximum stress and the maximum strain described above, and it is possible to produce a mycelium structure having further enhanced mechanical strength and flexibility.

Although the mycelium structure and the method for producing a mycelium structure according to the present disclosure have been described based on the preferred embodiment, the present disclosure is not limited thereto. For example, the mycelium structure according to the present disclosure may be what is obtained by replacing each unit of the embodiment described above with any component having a similar function, or what is obtained by adding any constituent to the embodiment described above.

The method for producing a mycelium structure according to the present disclosure may be one in which any desired process is added to the above embodiment.

### EXAMPLES

Next, specific examples of the present disclosure will be described, and the present disclosure is not limited thereto. A treatment and measurement in the following Examples were performed at room temperature (23° C.) for those not showing a temperature condition.

### [4] Production of Mycelium Structure

### [4-1] Example 1

First, a mycelium obtained by culture inoculum (Ganoderma lucidum) was prepared. Hyphae constituting the mycelium had an average diameter of 2.0 μm and a fiber density of 0.5 g/cm³.

Next, the mycelium prepared was immersed in a starch solution. A starch solution having a concentration of 20% by mass was used as the starch solution. After immersionat room temperature for 160 minutes, the mycelium was taken out from the starch solution.

Thereafter, the mycelium permeated with the starch solution was heated and pressurized to evaporate moisture. Heating and pressing conditions were 100.0° C. and 50.0 kPa, respectively.

As described above, the mycelium structure in Example 1 was obtained.

### [4-2] Examples 2 to 5 and Comparative Example 1

Mycelium structures in Examples 2 to 5 and Comparative Example 1 were obtained in the same manner as in Example 1 except that production conditions of the mycelium structure were changed as shown in FIG. 2 (Table 1). In Comparative Example 1, the mycelium was not permeated with a starch solution.

The mycelium structure obtained in Examples 2 to 5 had a density of 0.8 g/cm³ or more and 5.0 g/cm³ or less, and an average diameter of hyphae covered with starch was 2.0 μm or more and 20.0 μm or less.

### [5] Evaluation of Mycelium Structure

First, the mycelium structure was punched out to prepare a test piece. Next, a tensile test according to JIS P 8113:2006 was performed on the test piece using Autograph AGS-5kNX (produced by Shimadzu Corporation). A stress-strain curve indicating a relationship between a stress (MPa) applied by the tensile test and a strain (%) at that time was created, and an elastic region was specified.

### [5-1] Mechanical Strength

A maximum stress in the elastic region was evaluated based on the following evaluation criteria, and the mechanical strength was evaluated. The higher a numerical value of the maximum stress in the elastic region, the better the mechanical strength.
A: The maximum stress in the elastic region is 8.0 MPa or more and 40.0 MPa or less.
B: The maximum stress in the elastic region is 6.0 MPa or more and less than 8.0 MPa.
C: The maximum stress in the elastic region is 4.0 MPa or more and less than 6.0 MPa.
D: The maximum stress in the elastic region is 2.0 MPa or more and less than 4.0 MPa.
E: The maximum stress in the elastic region is less than 2.0 MPa.

### [5-2] Flexibility

A maximum strain in the elastic region was evaluated based on the following evaluation criteria, and the flexibility was evaluated. The higher a numerical value of the maximum strain in the elastic region, the better the flexibility.
A: The maximum strain in the elastic region is 1.5% or more and 4.0% or less.
B: The maximum strain in the elastic region is 1.0% or more and less than 1.5%.
C: The maximum strain in the elastic region is 0.5% or more and less than 1.0%.
D: The maximum strain in the elastic region is less than 0.5%.

These evaluation results are summarized in Table 1 together with the production conditions of the mycelium structures in Examples and Comparative Example.

As is clear from Table 1, the mycelium structures in Examples had excellent mechanical strength and flexibility. In contrast, in the mycelium structures in Comparative Example, satisfactory results were not obtained.

The mycelium structure was produced in the same manner as in Examples 1 to 5 except that in the drying step, a heating temperature was changed variously within a range of 80.0° C. or higher and 120.0° C. or lower and a pressurization condition was changed within a range of 40.0 kPa or more and 230.0 kPa or less. When these mycelium structures were evaluated in the same manner as described above, similar results as described above were obtained.

A mycelium structure was produced in the same manner as in Examples 1 to 5 except that, in the mycelium preparation step, an average diameter of hyphae constituting the mycelium was changed variously within a range of 0.2 μm or more and 4.0 μm or less, and a fiber density of the hyphae constituting the mycelium was changed variously within a range of 0.1 g/cm³ or more and 0.8 g/cm³ or less. When these mycelium structures were evaluated in the same manner as described above, similar results as described above were obtained.

What is claimed is:

1. A mycelium structure comprising:
a mycelium formed of hyphae connected in a three-dimensional shape; and
starch covering the hyphae.

2. The mycelium structure according to claim 1, which has a density of 0.8 g/cm³ or more and 5.0 g/cm³ or less.

3. The mycelium structure according to claim 1,
wherein an average diameter of the hyphae covered with the starch is 2.0 μm or more and 20.0 μm or less.

4. The mycelium structure according to claim 1,
wherein in a stress-strain curve indicating a relationship between a stress (MPa) applied by a tensile test and a strain (%) at that time, a maximum stress in an elastic region is 2.0 MPa or more and 40.0 MPa or less.

5. The mycelium structure according to claim 1,
wherein in a stress-strain curve indicating a relationship between a stress (MPa) applied by a tensile test and a strain (%) at that time, a maximum strain in an elastic region is 0.5% or more and 4.0% or less.

6. A method for producing a mycelium structure, comprising:
a permeation step of causing a starch solution to permeate into a mycelium obtained by culture; and
a drying step of drying the mycelium permeated with the starch solution.

7. The method for producing a mycelium structure according to claim 6, wherein
in the drying step, the mycelium permeated with the starch solution is heated at 80.0° C. or higher and 120.0° C. or lower.

8. The method for producing a mycelium structure according to claim 6, wherein
in the drying step, the mycelium permeated with the starch solution is pressurized at 40.0 kPa or more and 230.0 kPa or less.

9. The method for producing a mycelium structure according to claim 6, wherein
an average diameter of hyphae constituting the mycelium is 0.2 μm or more and 4.0 μm or less.

10. The method for producing a mycelium structure according to claim 6, wherein
a fiber density of hyphae constituting the mycelium is 0.1 g/cm³ or more and 0.8 g/cm³ or less.

11. The method for producing a mycelium structure according to claim 6, wherein
a starch concentration in the starch solution is 3.0% by mass or more and 30.0% by mass or less.

12. The method for producing a mycelium structure according to claim 11, wherein
the starch concentration in the starch solution is 15.0% by mass or more and 25.0% by mass or less.
